(19) United States
(12) Patent Application Publication
(10) Pub. No.: US 2021/0355098 A1
(43) Pub. Date: Nov. 18, 2021

(54) Methods For Extracting Constituents From Plant Material and Apparatus and Products Thereof

(71) Applicant: HURON BOTANICALS, LLC, Milford, MI (US)

(72) Inventors: Paul J. Kerry, Burtchville, MI (US); Brad J. Maurer, Burtchville, MI (US)

(21) Appl. No.: 17/173,193

(22) PCT Filed: Aug. 12, 2019
(86) PCT No.: PCT/US2019/000038

§ 371 Date: Feb. 10, 2021

Related U.S. Application Data
(60) Provisional application No. 62/717,615, filed on Aug. 10, 2018.
(60) Provisional application No. 62/791,391, filed on Jan. 11, 2019.

(51) Int. Cl. C07D 311/80; B01J 20/10; B01J 20/18; B01J 20/14; B01J 20/28; B01J 20/286; B01J 20/283; C11B 7/00; B01D 11/02; B01D 39/06

(52) U.S. Cl. CPC C07D 311/80; B01J 20/103; B01J 20/18; B01J 20/14; B01J 20/28052; B01J 20/286; B01D 2239/0407; C11B 7/00; B01D 11/0288; B01D 11/0292; B01D 39/06; B01J 2220/42; B01J 2220/52; B01J 20/283

(57) ABSTRACT

A closed loop extraction process contains a filter assembly 20 for vacuum filtration of an extraction solvent 50 after it has extracted desired constituents from a plant material. High purity products formed from the process are also provided. A closed loop extraction system 10 contains a filter assembly 20 for filtering an extraction solvent 50 and extract prior to collection of desired products within a collection vessel 34. A filter assembly 20, used in the aforementioned process and system 10, provides a novel enhancement in the current strategies to extract active ingredients from plant materials 52.

### CROSS-REFERENCE TO RELATED APPLICATIONS

The present application claims the benefit of U.S. Provisional Patent Application No. 62/717,615 having a filing date of Aug. 10, 2018, and, U.S. Provisional Patent Application No. 62/791,391 having a filing date of Jan. 11, 2019, the teachings of both applications each herein incorporated by reference in their entirety. The present application is a continuation-in-part of, and also claims priority to and the benefit of, co-pending International Patent Application Serial No. PCT/US19/00038 having a filing date of Aug. 12, 2019.

### FIELD OF THE INVENTION

The present application relates to improved and novel methods for extracting active and/or desired ingredients from plant material, and the products formed therefrom. The application also relates to improved and novel methods for extracting active and/or desired ingredients from plant material, and the equipment related thereto.

### BACKGROUND OF THE INVENTION

With the propagation and legalization of the hemp and cannabis industries throughout many areas in the world and in the United States, there is an ongoing effort to optimize the efficiency of removing and isolating desired products from these plants. Additionally, there is also an ongoing effort to improve on the purity of end products formed from associated extraction processes.

To that end, it is also an ongoing challenge to improve the processes and methods of extraction of plant materials, not only by developing new processes, but also in retrofitting known processes with devices designed to improve the methods and products formed from the plant material.

Yet further, oftentimes it becomes necessary to dispose of moldy or corrupted plant material because it simply cannot be used given the quality criteria for medicinal items, food items, smoking items, and vaping items made with these materials. For example, damp or humid ambient conditions during packing, shipping, and/or storage may contribute to the unforeseen molding of plants or plant material. Or, pesticides typically used in the control of mites in certain plant crops may present levels of pesticide unacceptable within one or more of the industries mentioned above. Accordingly, it would be an improvement in the art to provide an enhanced method(s) or process(es), and the associated products-by-process, that reflect due concern for these and other challenges thereby providing quality products for this emerging market.

### SUMMARY OF THE INVENTION

In a first aspect of the invention, a product is provided that is formed by a closed loop extraction process comprising the steps of: packing an extraction vessel with a plant material and sealing the extraction vessel; pumping an extraction solvent into the extraction vessel; retaining the extraction solvent within the extraction vessel for a predetermined amount of time; drawing the extraction solvent from the extraction vessel into and through a filter under vacuum; drawing the extraction solvent after filtration into a collection vessel under vacuum; and drawing the extraction solvent from the collection vessel by vacuum to isolate the product in the collection vessel.

In a second aspect of the present invention, a closed loop solvent extraction process contains the steps of: packing an extraction vessel with a plant material and sealing the extraction vessel; pumping an extraction solvent into the extraction vessel; retaining the extraction solvent within the extraction vessel for a predetermined amount of time; drawing the extraction solvent from the extraction vessel into and through a filter under vacuum, whereby the extraction solvent is filtered; drawing the extraction solvent from the filter into a collection vessel under vacuum; and drawing the extraction solvent from the collection vessel by vacuum to isolate the product in the collection vessel.

In a third aspect of the invention, a closed loop solvent extraction system contains a pressurizable solvent reservoir; an extraction column adapted to be in fluid communication with the pressurizable solvent reservoir; a filter assembly adapted to be in fluid communication with the extraction column; a recovery vessel adapted to be in fluid communication with the filter assembly; and a vacuum pump adapted to reduce the pressure to draw the solvent from the extraction vessel through the filter assembly, and then through the recovery vessel.

In a fourth aspect of the invention, a filter assembly adapted to fluidly communicate with an extraction vessel and a recovery vessel of a closed loop extraction system contains: a filter cup; and a first filter media comprising silica. Other layers within the filter assembly may be provided including a second layer containing molecular sieve or celite, for example; and a third layer containing diatomaceous earth, for example. These and other aspects of the invention are elaborated on below in the Detailed Description of the Invention.

### BRIEF DESCRIPTION OF THE DRAWINGS

FIG. 1 is a schematic representation of an extraction column or vessel, filter, and recovery vessel of the present invention;

FIG. 2 is a schematic representation of an exemplary extraction and recovery system of the present invention, including a detailed diagram of the extraction vessel cover;

FIG. 3 is a schematic representation of a second exemplary extraction and recovery system of the present invention;

FIG. 4 is an exemplary filter of a process, in accordance with the present invention;

FIG. 5 is an exemplary filter cup, in accordance with the present invention;

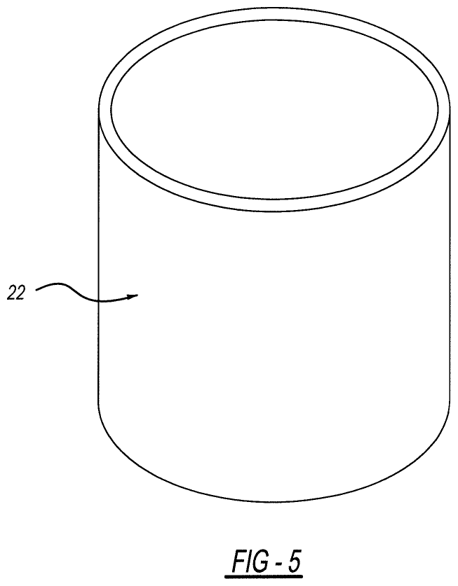

FIG. 6 is an exemplary filter polypad, in accordance with the present invention; and

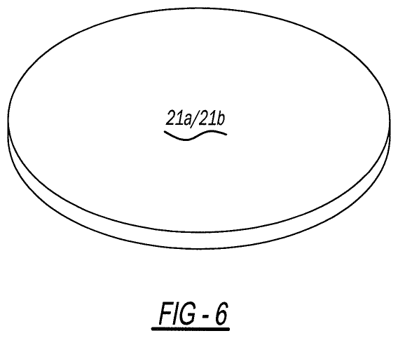

FIG. 7 is a schematic view of a distribution manifold in accordance with the present invention.

### DETAILED DESCRIPTION OF THE PRESENT INVENTION

The present invention contains an evaporative cooling system 10 for extracting oil and/or other constituents from oil-bearing plant parts, or from any other plants or parts of plants. An exemplary system that is known in the art is described in U.S. Pat. No. 9,399,180, the teachings of which are herein incorporated by reference. An upright stand 11 contains an extraction vessel 12. The extraction vessel 12 contains a hollow tube 12d having an open top 12e and an open bottom 12c. A peripheral top flange 14 extends about the circumference of the open top 12b, and, a peripheral bottom flange 16 extends about the circumference of the open bottom 12c.

A top cup 18 removably engages with the open top 12b, wherein the solvent is or may be substantially atomized before it is introduced to the extraction vessel 12. To that end, the top cup 18 has an open bottom 18a that is preferably the same size as the open top 12b. The top cup 18 therefore, is clamped or otherwise secured to the open top 12b by virtue of the flanges and the equivalent sizes of the top cup 18 and the open top 12b, to form a seal between the top cup 18 and the open top 12b.

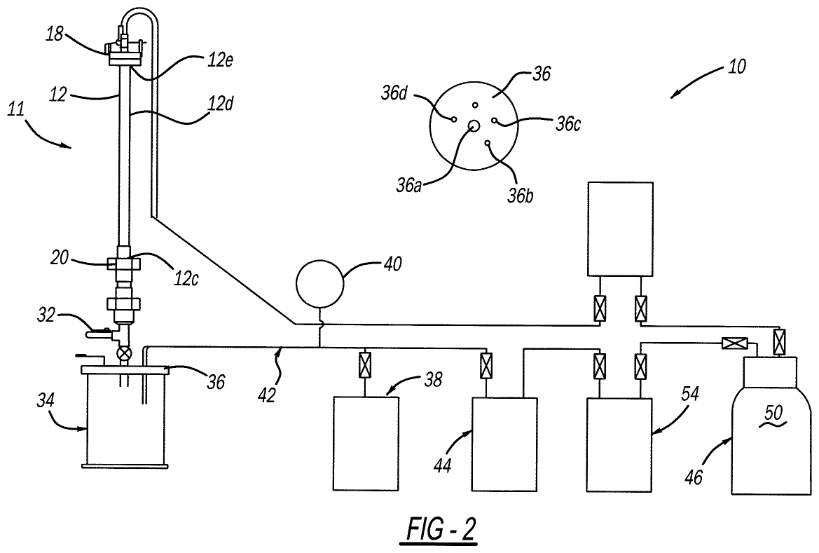

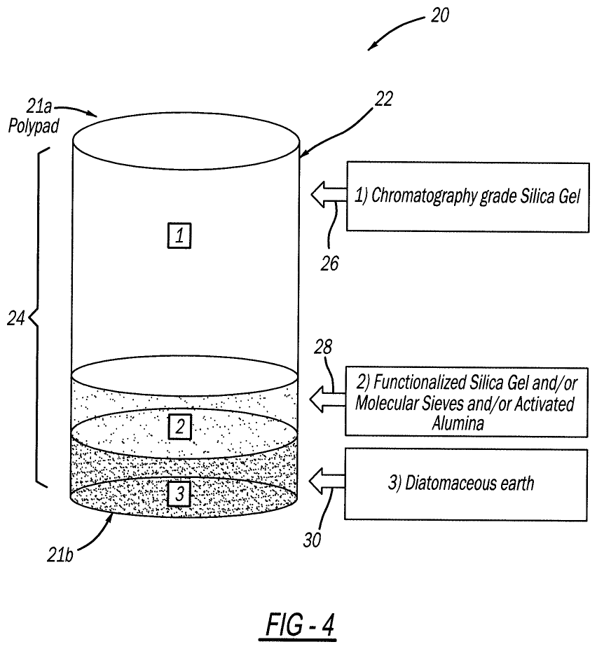

In accordance with the present invention, and as shown in FIGS. 2 and 4, a filter assembly 20 is removably fixed and sealed to the open bottom 12c of the extraction vessel 12, such that all fluids and effluent from the extraction vessel 12 flows into the filter assembly 20, for filtering thereof. As shown in FIG. 2, a filter cup 22 houses the inner constituents of the filter assembly 20. The filter cup contains a top retention pad or polypad 21a and a bottom retention or polypad 21b, whereby both function to retain the layers of filtration media within the cup 22, as described below, and yet permit fluid under pressure to flow therethrough. The polypads may be made from a polypropylene mesh or felt filter pad, or any other suitable material, with an exemplary pore size of about 10-100 microns. The filter cup or cartridge 22 is preferably designed to snugly fit within a standard industrial spool, whereby the spools may be sized at 1.5 inches, 3.0 inches, 6.0 inches, 12.0 inches, 24.0 inches, 36 inches in diameter, or larger, for example. A filter 24 is formed in layers within the cup 22. A first layer 26 may contain silica gel at about 200-400 mesh and more preferably at about 60-250 mesh, preferably chromatography grade silica gel, whereby the silica gel, occupying the top part of the filter cup 22, may occupy 50-100% of the total volume of the cup 22. It has been found that silica gel efficiently removes polar and non-polar components such as chlorophyll, carbohydrates, and protein from the extract, as well as unwanted materials from any insects, molds, or fungus. In another embodiment, the layer 26 may be provided at about 0.1-95% of the volume of the cup 22.

An optional second layer 28 may be provided depending on the pollutants and quality of the starting plant material. The second layer 28 may contain one or more of the following constituents: functionalized silica gel, molecular sieves, and activated alumina. These materials are chosen with regard to the particular materials to be filtered. If, for example, the plant material undergoing the extraction process is known to contain high levels of a particular chemical agent (for example, insecticide or pesticide) or heavy metal that would pass through the first layer 26, then one or more customized functional groups such as an amine, thiol, isocyanate, or other group that is combined with the silica gel may be used to remove the particular pollutant(s) in the second level 28 of filtration. Additionally, molecular sieves such as 3A, 4A, 5A, 13X, and so forth, may form the layer 28 or be added to adsorb ammonia, water, and/or waxes or other compounds that are retained within the molecular sieve. The removal of water and ammonia, for example, may be important to prevent corrosion of the equipment, thereby prolonging the life of the plant extraction system 10. Furthermore, activated alumina can remove contaminants such as heavy metals and water. Layer 28, optionally provided just below layer 26 with regard to fluid flow, may be provided at about 15-25% of the volume of the cup 22. In another embodiment, layer 28 may be provided at about 0.1 to 25% of the volume of the cup 22. In another embodiment layer 28 may be provided at about 0.1 to 5% of the volume of the cup 22.

Finally, an optional bottom third filtration layer 30 may be formed from diatomaceous earth, and is useful in removing any leaching filter media, as well as fine particulates. Layer 30 may be provided at about 10-20% of the volume of the cup 22, and essentially may serve as a polishing filter of the extraction solvent just prior to the solvent flowing into the collection vessel below. In yet another embodiment, layer 30 may be provided at about 0.1 to 20% of the volume of the cup 22. In yet another embodiment, layer 30 may be provided at about 0.1 to 5% of the volume of the cup 22. Layers 26 and 28 may be interchanged based on the desired end product. Yet further, the change of order and content of the layers 26 and 28 may change the order of removal of pollutants like pesticides, heavy metals, and molds, thereby affecting the overall remediation effort, and/or, with a cleaner product, the composition of the final product. All of the filter materials may be purchased from companies such as Silicycle, Sigma-Aldrich, or other suitable suppliers. An exemplary filter assembly 20 contains about 25-95% by volume of silica (standard chromatography grade of about 60-250 microns) and about 5-25% by volume of Celite (diatomaceous earth).

A valved connector 32, containing one or more ball valves for example, is removably sealed to the bottom of the filter cup 22 and to the top of a collection vessel 34, as described below. The valved connector 32 provides a conduit between the filter cup 22 and the collection vessel 34, and may be used to affect a flooding of the material spool or extraction vessel 12 by preventing and/or controlling fluid flow from the filter assembly 20 into the collection vessel 34.

A collection vessel 34 in operation fluidly communicates with and is removably sealed to the bottom of the filter cup 22, and thereby directly fluidly communicates with the filter assembly 20, and indirectly fluidly communicates with the extraction vessel 12. As shown in FIG. 2 and the Figures, a lid 36 is removably sealed to the top of the collection vessel 34. A column port 36a may be centrally positioned through the lid 36, thereby facilitating fluid flow from the filter assembly 20 into the collection vessel 34. A vacuum pump port 36b also extends through the lid 36, for establishing a vacuum within the collection vessel 34, and overall, within the system 10. A vacuum pump gauge port 36c, for receipt of a vacuum pump gauge 40, may be used to measure the vacuum of the system 10. A return line port 36d also extends through the lid for connection of a return vapor line 42. The collection vessel 34, in further accordance with the present invention, is preferably maintained at a temperature ranging from −40 C to 50 C, and more preferably from −5 C to 0 C.

A vacuum pump 38 is operably connected to the vacuum pump port 36b for establishing a vacuum within the system 10. A typical steady state pressure of −10 to 200 psi, as measured within the extraction vessel 12, may be maintained in the extraction vessel 12 by pressurizing the flow from the recovery tank 46 to the top cup 18, and also, by controlling the release of the pressurized solvent/extract from the filter assembly 20 through the valve 32 and into the collection vessel 34.

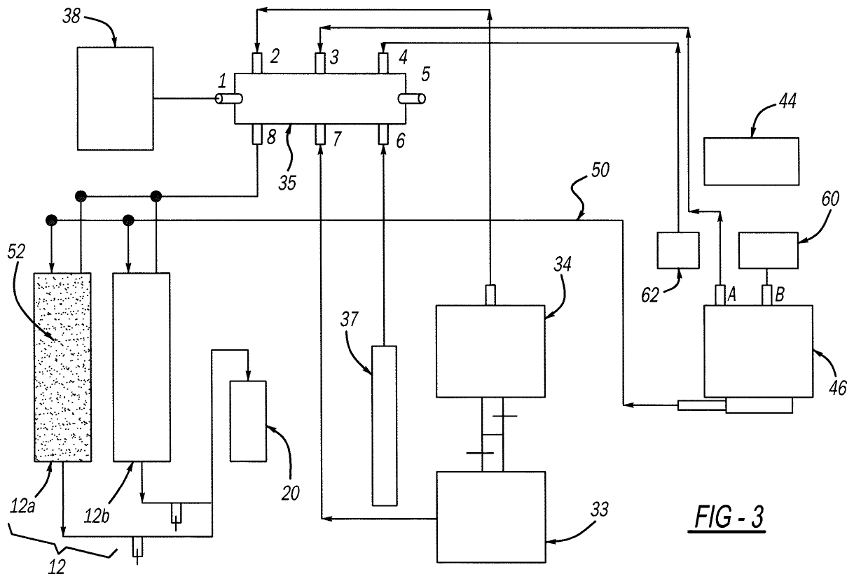

A bottom portion 33 is removably fixed to the collection vessel 34 for collection of plant product yield or desirable products. Additionally, in one embodiment, a window or reticle permits viewing of the interior of the bottom portion to visibly assess the amount of product yield and also the remaining butane in the bottom of the collection vessel. As the evacuation process continues over time, relative to one extraction vessel for example. As shown in FIG. 3, bottom portion 33 may fluidly communicate with the upper portion of the collection vessel 34 vis a vis the connecting valves 39. Shutting the valves 39 permits removal of the bottom portion 33 for removal of the process product yield while the evacuation process continues in the rest of the system 10, and thereby isolates the bottom portion 33 to permit continuation of evacuation from one extraction vessel 12a to the other extraction vessel 12b, and any other extraction vessels that may also fluidly communicate with the system 10.

A first recovery pump 44 is operably connected to the vapor recover line 42, which in turn fluidly communicates with the collection vessel 34, by virtue of the return line port 36d of the lid 36. The first recovery pump 44 essentially evacuates the collection vessel 34 of the extracting solvent, while leaving the product yield within the collection vessel 34. A compressor pump may, for example, be used as the first recovery pump 44.

A condenser 54 fluidly communicates with the first recovery pump 44, whereby the extracted solvent is chilled prior to returning it to its storage bottle/recovery tank 46. The extraction solvent first recovery tank, reservoir, or storage bottle 46 may contain any type of solvent 50 that is typically used in extraction processes. Any suitable alkane, for example, such as iso-butane, butane, propane, pentane, hexane and so forth may be used in the present system 10. Alkanes are preferred, and iso-butane is particularly preferred. It is preferable that the storage bottle 46 also be maintained at a relatively cooler temperature to thereby provide a cooled or cold liquid solvent prior to flooding the extraction vessel 12 with the solvent, at the beginning of the extraction process. A “cold solvent” is understood to be a solvent that is a liquid solvent at a pressure and temperature that results in the solvent being a liquid at the respective pressure and temperature.

In operation, and with reference to the figures including FIGS. 2-4, the extraction vessel or column 12 is first packed with a desired plant material 52, and then the extraction vessel 12 is installed within the system 10, in a sealed state. A pre-chiller 48 may fluidly communicate with an outlet valve of the storage bottle 46, thereby further chilling the extraction solvent 50, prior to pumping it into the extraction vessel 12 through the top cup 18. The vacuum pump 38 may then be actuated to bring the evaporative cooling system 10 under vacuum prior to the next extraction step.

After preferably bringing the system 10 under vacuum, the solvent 50 such as butane for example, is then pumped into the extraction vessel 12, at a rate of about 4-8 pounds of solvent per pound of starting plant material. The solvent 50 is pumped or provided from pressurized extraction solvent reservoir 46 and floods the extraction vessel 12 to begin the extraction of the desired constituents from the plant material 52. As the solvent 50 permeates the plant material 52, the solvent 50 soaks the plant material 52 and may be retained within the extraction vessel 12 for a desired amount time, with an exemplary retention range of 0.5 to 4.0 hours, depending on what constituents are being isolated from the material 52 within the extraction vessel 12. Importantly, as shown in FIG. 3 for example, the operative extraction vessel 12 (12a and/or 12b) directly fluidly communicates with the butane or solvent storage tank 46. In another embodiment, one or more extraction vessels or columns 12a and 12b may be used to provide a system 10 that concurrently provides extraction of the plant material 52 within a first extraction vessel 12a and concurrent evacuation (explained below) of a second extraction vessel 12b.

Once the solvent 50 has soaked the plant material 52 for a desired amount of time, and extracted a substantial amount of the desired constituents from the plant material 52, the solvent 50 is then pumped/drawn through the filter assembly 20, for purification of the solvent and extract. Accordingly, the first recovery pump 44 is actuated once the extraction process in extraction vessel 12a or 12b is terminated and the filtration step begins. As described herein, filtering the extract solution or solvent 50, prior to isolation of the desired products, removes undesirable contaminants such as mold, pesticides, and/or plant constituents that otherwise would contaminate the final product(s).

Upon leaving the filter assembly 20, the solvent and extract is received within the collection vessel 34, to thereby provide a purified product in a one-step process, in accordance with the present invention. To that end, the solvent 50 is pumped from the collection vessel 34 while the product remains in a bottom portion of the collection vessel 34. Stated another way, the extraction solvent is evacuated from the collection vessel 34 and returned through the recovery pump 44, through the condenser 54, and into the first solvent reservoir/recovery tank 46 for subsequent use in another batch process.

The exemplary process shown in FIG. 3 augments the exemplary process of FIG. 2. During the extraction process, the pressure may occasionally increase within the first recovery tank 46. It has been found that as the pressure of the tank approaches 50 psi, the backpressure to the system 10 may interfere with the evacuation of the butane from the extraction column 12a or 12b and the collection vessel 34. Accordingly, a second recovery pump 60 in fluid communication with the first recovery tank 46 may then be actuated to relieve butane pressure from first recovery tank 46, by pumping the relatively high-pressure butane into a second recovery tank 62 for the storage of butane.

As also shown in FIG. 3, it can be seen that butane stored in the second recovery tank 62 can also be pumped to the manifold 35 for transfer to one or both of the extraction columns 12a and 12b during the extraction step, to thereby ensure a sufficient head over any remaining extract solvent within one or both of the extraction columns 12a and 12b. As shown in FIG. 3, manifold 35 has a plurality of valves or ports 1-7 all adapted to be in fluid communication with a central plenum 35a within the manifold 35. Accordingly, various fluid flow schemes may be accommodated through appropriate valving between the manifold 35 and two or more other constituents of the system 10. For example, second recovery tank 62 may be valved to fluidly communicate with manifold 35 and one or both of the extraction columns 12a and 12b, to thereby augment or replace the flow of nitrogen into the columns during an extraction step, for example. By pumping butane from the first recovery tank 46 through second recovery pump 60 into second recovery tank 62 and then into the manifold 35, butane gas from second recovery tank 60 can be pumped into an extraction column to keep the column head at a pressure level that enhances the evacuation of the extraction solvent from the columns 12a or 12b.

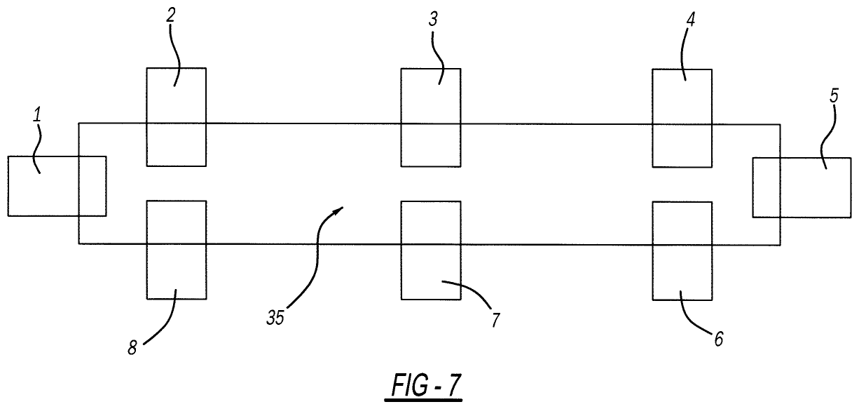

The aforementioned manifold 35 is shown in FIG. 7. As shown in FIG. 3 and FIG. 7, the manifold 35 may affect fluid communication between various constituents of the system 10 during various steps of the overall process. For example, with reference to FIGS. 2 and 3 also, when bringing the overall closed loop system 10 to vacuum conditions, the fluid flow lines and valves of the various lines from and associated with various constituents of the system 10 are opened or closed as necessary to facilitate creating a vacuum across the system, including the lines associated with the extraction vessel or columns 12a and 12b, the filtration assembly 20, the collection vessel 34, the fluid flow lines that provide fluid communication between these constituents, and, the fluid flow lines that provide fluid flow communication between the butane or extraction solvent reservoir 46 and the nitrogen tank, and the rest of the system 10, for example.

Related thereto, and as schematically shown in FIG. 3, for example only and not by limitation, valve A of solvent extraction solvent (e.g. butane) reservoir/recovery tank 46 fluidly communicates with valve 3 of the manifold 35 for distribution to valve 8, to provide gaseous butane or extraction solvent to the extraction columns 12a or 12b (generically characterized as reference number 12), thereby providing a pressure head during the evacuation step as described herein, thereby “squeezing” the extraction solvent from the packed material 52 in the extraction column 12. Yet further, valve 4 of the manifold 35 fluidly communicates with the second recovery tank 62 to again provide gaseous butane or extraction solvent to the extraction column 12 vis a vis valve 8, again for purposes of creating a head within extraction column 12 during the evacuation step. Yet further, valve 6 of the manifold 35 fluidly communicates with the nitrogen source that again may also fluidly communicate with valve 8 for purposes of creating a head in extraction column 12 during the evacuation step. Yet further, valve 7 of manifold 35 may fluidly communicate with bottom portion 33, whereby as the amount of butane or extraction solvent appears to wane or decrease over time, as viewed through the optical window of the bottom portion 33 or otherwise automatically or manually monitored as known in the art, residual amounts of solvent 50 may be vacuumed through manifold 35, through the vacuum pump connected at valve 1, for example. In this way, a product-by-process is facilitated without the need to further dry and cook the final product found in collection vessel 34 (i.e. bottom portion 33).

Yet further, valves 1, 2, 7, and 8 may fluidly communicate with a vacuum pump to facilitate bringing the system 10 under vacuum prior to the soaking step.

Other fluid flow arrangements facilitated through system 10 by and through manifold 35 are contemplated, and the fluid flow arrangements discussed above should not be considered and are not meant to be limiting.

In one aspect of the present invention, a product may be formed by a closed loop extraction process containing the following steps: packing an extraction vessel with a plant material and sealing the extraction vessel; pumping an extraction solvent containing a hydrocarbon-based solvent into the extraction vessel; retaining the extraction solvent within the extraction vessel for a predetermined amount of time to produce one or more extracts from the plant material within the extraction solvent; drawing or evacuating the extraction solvent from the extraction vessel into a filter under vacuum, wherein the filter contains silica and is located immediately downstream and after the extraction vessel—(the clause or term “immediately downstream” is meant to indicate that the filter follows the extraction column before any other constituent of the system); filtering the extraction solvent containing the one or more extracts after retaining the extraction solvent within the extraction vessel; after filtration, drawing the extraction solvent into a collection vessel under vacuum; and drawing the extraction solvent from the collection vessel to isolate the product in the collection vessel wherein the closed-loop process is a cold solvent process.

In yet another aspect of the invention, a closed loop solvent extraction process may contain the following steps: packing an extraction vessel with a plant material and sealing the extraction vessel; pumping an alkane-based cold extraction solvent into the extraction vessel wherein the term “alkane-based” is meant to only include non-substituted alkanes, or alkanes containing only hydrogen and carbon; retaining the extraction solvent within the extraction vessel for a predetermined amount of time to extract from the plant material and produce at least one extract contained within the extraction solvent; drawing the cold extraction solvent from the extraction vessel directly into and through a silica filter under vacuum, whereby the extraction solvent containing at least one extract is filtered; drawing the cold extraction solvent from the filter into a collection vessel under vacuum; and drawing the extraction solvent from the collection vessel by vacuum to isolate the extract product in the collection vessel.

Again, although not by limitation, another aspect of the invention includes a filter assembly containing: a filter cup; and a first filter media layer comprising chromatography-grade silica, for filtering an alkane-based solvent containing an extract, wherein the filter assembly is adapted to fluidly communicate with an extraction vessel and a recovery vessel of a closed loop alkane-based solvent extraction system.

Other features of the invention as discussed herein, or as known in the art, may also be integrated in the various aspects of the invention.

The following Examples exemplify but do not limit additional aspects of the present invention.

### Example 1

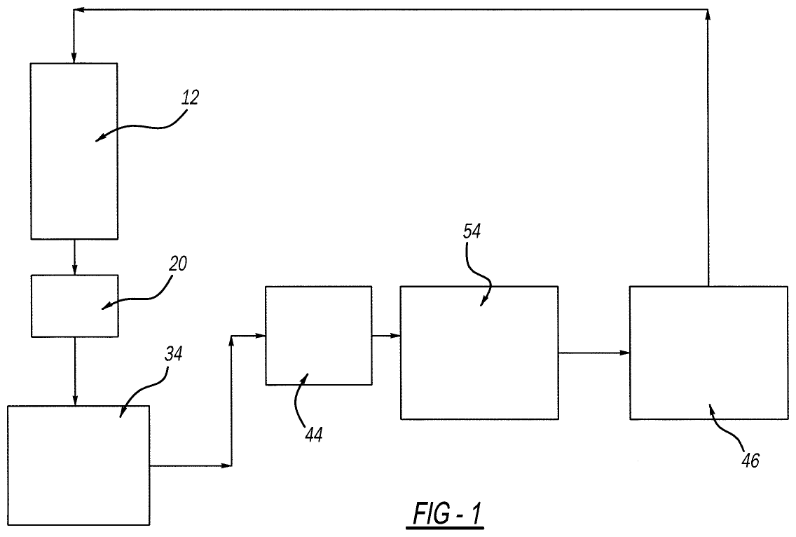

For medicinal purpose, one pound (dry basis) of a normal or fresh plant material 52, such as cannabinoid trim, is packed within the extraction vessel 12. The system 10 is then assembled as described above and as shown in FIGS. 1-3, for example. The vacuum pump 38 is actuated to bring the system 10 under vacuum, as described herein. About eight pounds of butane solvent 50 is introduced into the feed line in fluid communication with the top cup 18 from solvent reservoir and recovery tank 46. The solvent 50 is first introduced into the chiller 48 and exits the chiller at a temperature of about −20 C to −10 C. As the chilled extraction solvent or butane 50 is introduced to extraction column 12, the packed material 52 is soaked with the solvent 50 for a residence time ranging from about 30 minutes to four hours. During the residence soak time, the extraction column 12 is isolated from the rest of the system 10 by appropriately valving off the column(s) 12 from the rest of the system 10. The steady state pressure of the extraction vessel 12 is about −10 to 200 psi, prior to entering the filter assembly 20. The steady state temperature within the extraction vessel is about −20 C to about 20 C. As the residence soak time within extraction column 12 is completed, the system 10 is then valved to open the various components or constituents of the system to provide fluid communication between them as described herein. Accordingly, the first solvent recovery pump 44 is actuated and begins the evacuation process by drawing the extraction solvent from the extraction column 12 through the filter assembly 20. In this exemplary embodiment, the filter assembly 20 contains by volume about 5 to 95% silica and about 5 to 25% by volume diatomaceous earth. As the solvent 50 and resultant extract from the column 12 flows through the system 10, filter 20, and into the collection vessel 34, the separated solvent 50 is then pumped back into the condenser 54, with an input temperature range of about 10 C to 80 C, and a condenser exit temperature of about 0 C to 15 C. The solvent 50 is then pumped back into the first recovery tank/reservoir 46 for use in the next batch process.

The process may require from four to twenty-four hours. A product yield of about 0.1 to 25% by weight, based on the dry weight of the starting plant material 52, is collected. The purity of the product yield is estimated to be about 50-95% THCa/Delta 9 THC; and 5-20% terpenes, plant waxes, and residual solvents, the percents taken by weight of the total product.

In accordance with the present invention, the product yield from this exemplary process is harvested from the collection vessel and may be left exposed to the ambient environment for a desired amount of time, to air or otherwise permit any minimal potential solvent residue to volatilize, thereby resulting in a one-pot process.

### Example 2

For medicinal purpose, one pound (dry basis) of corrupted plant material 52, such as cannabinoid trim, is packed within the extraction vessel 12a or 12b. The term “corrupted” is exemplified by plant material that may contain fungus, mold, pesticides, insects, and so forth. The system 10 may then be assembled as described above and as shown in FIGS. 1-3. The vacuum pump 38 is actuated to bring the system 10 under vacuum prior to soaking the packed extraction column 12a or 12b. About eight pounds of butane solvent 50 is then introduced into the feed line in fluid communication with the top cup 18. The solvent 50 is first introduced into the chiller 48 and exits the chiller at a temperature of about −20 C to −10 C. The plant material 52 is then soaked with butane for about thirty minutes to four hours. The steady state pressure of the extraction vessel 12 during the soaking step may fluctuate between about −10 to 200 psi. The steady state temperature within the extraction vessel during the soaking step is about −20 C to about 20 C.

Once the soaking or extraction step is terminated, and prior to entering the filter assembly 20, the first recovery pump 44 is actuated. Accordingly, during the extraction process, nitrogen and/or gaseous extraction solvent 50 may be provided to the respective extraction column 12a or 12b to maintain a pressure head of about 50 psi or more in the extraction column. As shown in FIG. 3, nitrogen may be provided through the distribution manifold 35 from a pressurized nitrogen tank 37, for example.

The filter assembly contains by volume about 75% silica and about 25% by volume diatomaceous earth. As the solvent 50 and resultant extract from the column 12 flows through the system 10, filter 20, and into the collection vessel 34, the separated solvent 50 is then pumped back into the condenser 54, with an input temperature range of about 10 C to 80 C, and a condenser exit temperature of about 0 C to 15 C. The solvent 50 is then pumped back into the first recovery tank/reservoir 46 for use in the next batch process.

The process may require from four to eighteen hours. A product yield of about 1 to 15%, based on the dry weight of the starting plant material, is collected. The purity of the product yield is about 50 to 90% THCa/Delta 9 THC; and about 5 to 20% terpenes, plant waxes, and residual solvents, the percents taken by weight of the total product.

In accordance with the present invention, the product yield from this exemplary process is harvested from the collection vessel and may be left exposed to the ambient environment for a desired amount of time, to air or otherwise permit any minimal and potential solvent residue to volatilize for example, thereby resulting in a one-pot process.

### Example 3 (Product 1)

The system of Example 1 was assembled and initiated with the stated amount of starting material (cannabis flower and trim) and using butane or any other suitable hydrocarbon solvent, whereby the concentration temperature in the collection vessel 34 ranged from −50 C to 30 C to produce a concentrated extract. Once removed from the collection vessel 34, the extract contained white to off-white cannabinoid solids and plant wax solids in a mixture of oily residue. The oily residue was found to contain colored terpenes that were yellow, red, and orange in color, and also found to contain extractable flavonoids. An analysis of the product yield indicated relatively high THCA levels and relatively low delta-9 THC levels (substantially equivalent to the amount of Example 3). The proprietary filter notably contained no molecular sieves 13x, and the product yield therefore also contained plant waxes that are believed to contribute to the precipitation of solids. Terpenes are desirably preserved based on the relatively lower collection temperature. The product was also found to smell like cannabis flower with enhanced terpene aroma. When producing a product of the present example, it has been found that “live” or “freshly cut” cannabis flowers or trim contributed to relatively higher yields of THCA and terpenes. Based on this example, it is also believed that product stability is enhanced due to solidified nature of the THCA, thereby reducing degradation of the cannabinoids.

### Example 4 (Product 2)

The system of Example 1 was assembled and initiated with the stated amount of starting material (cannabis flower and trim) and using butane or any other suitable hydrocarbon solvent, whereby the concentration temperature in the collection vessel 34 ranged from −50 C to 30 C to produce a concentrated extract. Once removed from the collection vessel 34, the extract contained white to off-white cannabinoid solids and plant wax solids in a mixture of less viscous liquid than the oily residue of Example 3. The relatively less viscous liquid was found to contain colored terpenes that were yellow, red, and orange in color, and also found to contain extracted flavonoids. An analysis of the product yield indicated relatively high THCA levels and relatively low delta-9 THC levels (about 1-10 weight percent). The proprietary filter notably contained molecular sieves 13x at about 5 to 50% percent by volume of the filter cup (preferably 30 to 40 volumetric % of the filter cup), and the product yield therefore also contained substantially less plant waxes at about 5 to 10 weight percent of the product yield, that are believed to contribute to the precipitation of solids. Terpenes are desirably preserved based on the relatively lower collection temperature. The product was also found to smell like cannabis flower with enhanced terpene aroma. When producing a product of the present example, it has been found that “live” or “freshly cut” cannabis flowers or trim contributed to relatively higher yields of THCA and terpenes. Based on this example, it is noted that a distinguishable type of THCA crystals precipitated from the mixture, as compared to Example 3.

### Example 5 (Product 3)

The system of Example 1 was assembled and initiated with the stated amount of starting material (cannabis flower and trim) and using butane or any other suitable hydrocarbon solvent, whereby the concentration temperature in the collection vessel 34 ranged from a relatively high temperature of 30 C to 140 C to produce a concentrated extract. Once removed from the collection vessel 34, the extract contained viscous cannabinoid residue mainly containing delta-9 THC with low THCA levels and minimal or no solids. The viscous residue was found to contain colored terpenes that were yellow, red, and orange in color. An analysis of the product yield indicated relatively low THCA levels and relatively high delta-9 THC levels. The proprietary filter notably contained no molecular sieves 13x, and the product yield therefore also contained plant waxes (about 0.1-10 weight percent) that are believed to contribute to the precipitation of solids. Minimal to no solids were therefore observed in the product yield. Terpenes are less preserved (as compared to Examples 3 and 4) based on the relatively higher collection temperature and drying, which is believed to remove or at least substantially reduce the amount of high boiling temperature liquids (e.g., greater than the boiling temperature of butane) such as terpenes and flavonoids in the product yield. The final product is a crumbly or loose substance that presents as a crumbly product similar to crystallized honey. If desired, flash film drying can produce a product that presents as a glassy or shale-like substance, similar to a thin rock candy. In this process, a concentrate liquid is poured onto a silicon coated or other non-stick paper and then dried at ambient temperatures of about 25 C for about 4-12 hours.

### Example 6 (Product 4)

The system of Example 1 was assembled and initiated with the stated amount of starting material (cannabis flower and trim) and using butane or any other suitable hydrocarbon solvent, whereby the concentration temperature in the collection vessel 34 ranged from a relatively high temperature of 30 C to 140 C to produce a concentrated extract. Once removed from the collection vessel 34, the extract contained viscous cannabinoid residue mainly containing delta-9 THC with low THCA levels. The viscous residue was found to contain colored terpenes that were yellow, red, and orange in color. An analysis of the product yield indicated relatively low THCA levels and relatively high delta-9 THC levels. The proprietary filter notably contained molecular sieves 13x, and the product yield therefore also contained relatively minimal or no plant waxes that are believed to contribute to the precipitation of solids. Terpenes are less preserved (as compared to Examples 3 and 4) based on the relatively higher collection temperature and drying, which is believed to remove or at least substantially reduce the amount of high boiling temperature liquids (e.g., greater than the boiling temperature of butane) such as terpenes and flavonoids in the product yield.

### Example 7 (Product 5)

The product of Example 3 was further filtered via vacuum/pressure filtration apparatus, and purified by rinsing with a suitable solvent such as an alkane selected from butane, propane, and/or hexane. In the rinsing process, one to five volumes of solvent may be run through the filter, wherein the term “volume” is defined as an equivalent of the volume of the starting material (the volume of the one pound of a plant material as stated in Example 1). Accordingly, one to five volumes of a suitable solvent such as an alkane selected from butane, propane, and/or hexane may be utilized in the rinsing step. The product yield contained white to off-white cannabinoid solids including over 80% by weight of THCA, about 1-10% by weight of delta-9 THC, and plant waxes of about 1-10 weight percent. The product yield was found to be substantially free of extracted terpenes and other materials. Stated another way, the product yield primarily contained THCA with low delta-9 THC levels, that may also contain plant waxes.

### Example 8 (Product 6)

The filtrate of the product of Example 3 primarily contains non-solid cannabinoids with colored terpenes (red, yellow, and orange). The filtrate (concentrated filtrate and rinse) presents a liquid from extracted cannabis mainly containing terpenes, flavonoids, and other cannabinoid materials extracted from cannabis at temperatures of −50 to 100 C.

### Example 9 (Product 7)

The product of Example 7 was decarboxylated to present a viscous liquid substantially free of extracted terpenes (and other materials such as plant waxes). Decarboxylation, or removal of the —COOH— groups on the extract by heating from 80 to 140 degrees C., resulted in the removal of terpenes and other materials such as plant waxes. The heating step was monitored by gas chromatography sampling to prevent product degradation (i.e., greater amounts of delta-9 THC): a sample was taken about every fifteen minutes and evaluated for the gradual increase of delta-9 THC and the gradual decrease of THCA. The goal was to minimize the amount of delta-9 THC being formed by controlling the time and temperature during the periodic sampling in the heating step. In this way, the amount of THCA and delta-9 THC could be controlled in the final product. The use of catalytic amounts of biologically derived compounds may be used to enhance conversion and prevent product degradation, thereby enhancing the ease of the decarboxylation.

### Example 10 (Product 8)

The product of Example 4 was further filtered via vacuum/pressure filtration apparatus, and purified by rinsing with a suitable solvent such as an alkane selected from butane, propane, and/or hexane. In the rinsing process, one to five volumes of solvent may be run through the filter, wherein the term “volume” is defined as an equivalent of the volume of the starting material (the volume of the one pound of a plant material as stated in Example 1). Accordingly, one to five volumes of a suitable solvent such as an alkane selected from butane, propane, and/or hexane may be utilized in the rinsing step. The product yield contained mostly THCA with relatively low amounts of delta-9 THC. The resulting solids were higher purity than the product yield of Example 9, with little or no precipitation of plant wax solids.

### Example 11 (Product 9)

The filtrate of Example 4, resulting from a filtration process as described in Example 7, contained substantially delta-9 THC, and other non-solid cannabinoids with colored terpenes (orange, red, and brown). The product yield is a liquid from extracted cannabis mainly containing terpenes, flavonoids, and other cannabinoid materials extracted from cannabis at temperatures of −50 to 100 C. The yield may contain THCA, delta-9 THC, CBD, and other cannabinoids depending on the type of cannabis extracted.

### Example 12 (Product 10)

The product of Example 10 was decarboxylated to present a viscous liquid substantially free of extracted terpenes (and other materials such as plant waxes). Decarboxylation, or removal of the —COOH— groups on the extract by heating from 80 to 140 degrees C., resulted in the removal of terpenes and other materials such as plant waxes. The heating step was monitored by gas chromatography sampling to reveal the composition constituents (i.e., greater amounts of delta-9 THC): a sample was taken about every fifteen minutes and evaluated for the gradual increase of delta-9 THC and the gradual decrease of THCA. The goal was to minimize the amount of THCA being formed by controlling the time and temperature during the periodic sampling in the heating step. In this way, the amount of THCA and delta-9 THC could be controlled in the final product. The product resulted in a relatively high delta-9 THC level or weight percent, a relatively low THCA level or weight percent, and less plant waxes that may have been contained in Example 9. The use of catalytic amounts of biologically derived compounds may be used to enhance conversion and prevent product degradation, thereby enhancing the ease of the decarboxylation.

### Example 13 (Mold-Laden Sample)

A system as described in Example 1 or Example 2 above was developed and operated. A plant material 52, corrupted with qualitatively high amounts of mold and qualitatively confirmed to have substantial amounts of mold (by smell and visual observation, for example), was packed into a column 12 in accordance with the present invention. The process was then operated as described in Examples 1 or 2, and as otherwise described herein. The resultant product, analyzed by known methods of High-Pressure Liquid Chromatography, was found to have no detectable amounts of mold in the product yield.

### Example 14 (Pesticide-Laden Sample)

A system as described in Example 1 or Example 2 above was developed and operated. A plant material 52, corrupted with qualitatively high amounts of pesticide (used to control mites for example) and qualitatively confirmed to have substantial amounts of pesticides (by smell and visual observation, for example), was packed into a column 12 in accordance with the present invention. The process was then operated as described in Examples 1 or 2, and as otherwise described herein. The resultant product, analyzed by known methods of High-Performance Liquid Chromatography, was found to have no detectable amounts of pesticides in the product yield.

References herein to the positions of constituents, for example “top,” “bottom,” “above,” “below,” etc., are merely used to describe the orientation of various elements in the FIGURES. It should be noted that the orientation of various elements may differ according to other exemplary embodiments, and that such variations are intended to be encompassed by the present disclosure.

In general, it will be understood that the foregoing descriptions of the various embodiments are for illustrative purposes only. As such, the various structural and operational features herein disclosed are susceptible to a number of modifications, none of which departs from the scope of the appended claims.

What is claimed is:

1. A product formed by a closed loop extraction process comprising the steps of:
packing an extraction vessel with a plant material and sealing the extraction vessel;
pumping an extraction solvent comprising a hydrocarbon-based solvent into the extraction vessel;
retaining the extraction solvent within the extraction vessel for a predetermined amount of time to produce one or more extracts from the plant material within the extraction solvent;
drawing or evacuating the extraction solvent from the extraction vessel into a filter under vacuum, wherein the filter comprises silica and is located immediately downstream and after the extraction vessel;
filtering the extraction solvent containing the one or more extracts after retaining the extraction solvent within the extraction vessel;
after filtration, drawing the extraction solvent into a collection vessel under vacuum; and
drawing the extraction solvent from the collection vessel to isolate the product in the collection vessel,
wherein said closed-loop process is a cold solvent process.

2. The product of claim 1 wherein the hydrocarbon-based solvent is selected from the group consisting of iso-butane, butane, propane, pentane, and hexane.

3. The product of claim 2 wherein the hydrocarbon-based solvent is iso-butane.

4. The product of claim 1 wherein the process further comprises the step of:
creating a vacuum throughout the system prior to pumping an extraction solvent into the extraction vessel.

5. The product of claim 1 wherein the process includes the steps of:
storing the extraction solvent drawn from the collection vessel in a first recovery tank; and
pumping extraction solvent from the first recovery tank into a second recovery tank.

6. A closed loop solvent extraction process comprising the steps of:
packing an extraction vessel with a plant material and sealing the extraction vessel;
pumping an alkane-based cold extraction solvent into the extraction vessel;
retaining the extraction solvent within the extraction vessel for a predetermined amount of time to extract from the plant material and produce at least one extract contained within the extraction solvent;
drawing the cold extraction solvent from the extraction vessel directly into and through a silica filter under vacuum, whereby the extraction solvent containing at least one extract is filtered;
drawing the cold extraction solvent from the filter into a collection vessel under vacuum; and
drawing the extraction solvent from the collection vessel by vacuum to isolate the extract product in the collection vessel.

7. The process of claim 6 wherein said cold extraction solvent is a liquid hydrocarbon.

8. The process of claim 6 wherein said cold extraction solvent is selected from the group consisting of hydrocarbon-based solvents.

9. The process of claim 8 wherein said hydrocarbon-based solvents are selected from the group consisting of alkanes.

10. The process of claim 9 wherein said hydrocarbon-based solvents are selected from the group consisting of iso-butane, butane, propane, pentane, and hexane.

11. A closed loop solvent extraction system comprising:
a pressurizable solvent reservoir containing a hydrocarbon-based solvent;
an extraction column adapted to be in fluid communication with said pressurizable solvent reservoir for producing an extract within said liquid hydrocarbon-based solvent;
a filter assembly adapted to be in fluid communication with said extraction column for filtering said extraction solvent, said filter assembly containing silica and said extraction solvent containing an extract flowing from said extraction column;
a collection vessel adapted to be in fluid communication with said filter assembly, for collection of said extraction product; and
a first recovery pump adapted to reduce the pressure to draw the solvent from the extraction vessel through the filter assembly, and then through the collection vessel and into a first recovery tank,
wherein said filter assembly immediately follows said extraction column, and, said hydrocarbon-based solvent is selected from the group consisting of iso-butane, butane, propane, pentane, and hexane.

12. The system of claim 11 further comprising a second extraction column in fluid communication with said pressurizable solvent reservoir, and further in fluid communication with said filter assembly for filtration of an extract and extraction solvent flowing from said second extraction column.

13. The system of claim 11 further comprising a removable portion of said collection vessel, for collection of a product yield.

14. A filter assembly comprising:
a filter cup; and
a first filter media layer comprising chromatography-grade silica, for filtering an alkane-based solvent containing an extract,
wherein said filter assembly is adapted to fluidly communicate with an extraction vessel and a recovery vessel of a closed loop alkane-based solvent extraction system.

15. The filter assembly of claim 14 wherein said chromatography-grade silica is provided at 200-400 mesh.

16. The filter assembly of claim 14 wherein said chromatography-grade silica is provided at 60-250 mesh.

17. The filter assembly of claim 14 wherein said alkane-based solvent is selected from iso-butane, butane, propane, pentane, and hexane.

18. The filter assembly of claim 14 further comprising a second layer containing molecular sieve.

19. The filter assembly of claim 16 further comprising a third layer containing diatomaceous earth.

20. The filter assembly of claim 14 further comprising an additional layer of functionalized silica.
